(19) United States
(12) Patent Application Publication
(10) Pub. No.: US 2023/0301523 A1
(43) Pub. Date: Sep. 28, 2023

(54) SYSTEMS AND APPARATUSES FOR PHYSIOLOGICAL AND PSYCHOLOGICAL PARAMETER MONITORING FROM A SUBJECT'S HEAD AND METHODS OF USE THEREOF

(71) Applicant: NEUROSTEER LTD., New York, NY (US)

(72) Inventor: Nathan INTRATOR, New York, NY (US)

(21) Appl. No.: 18/004,240

(22) PCT Filed: Jul. 8, 2021
(86) PCT No.: PCT/US2021/040782

§ 371 Date: Jan. 4, 2023

Related U.S. Application Data
(60) Provisional application No. 63/050,000, filed on Jul. 9, 2020.

(51) Int. Cl. A61B 5/0205; A61B 5/256; A61B 5/1455; A61B 5/349; A61B 5/00; A61B 5/372; A61B 5/024

(52) U.S. Cl. CPC A61B 5/0205; A61B 5/256; A61B 5/14553; A61B 5/349; A61B 5/721; A61B 5/746; A61B 5/372; A61B 5/6814; A61B 5/02416; A61B 2562/0219; A61B 5/4821; A61B 5/16

(57) ABSTRACT

A method includes receiving from a psychological and physiological sensing (PPS) device worn on a subject's head, sensor data from sensors fixed to the PPS device. The sensors may include a left-temple photoplethysmography (PPG) sensor, a right-temple PPG sensor, and an electroencephalogram (EEG) sensor coupled to the subject's head. The right and left temple PPG sensors are configured to detect pulsating blood flow in blood vessels proximal to a left and right temple region. Pulse morphology data of pulses related to the pulsating blood flow from the left-temple PPG signal and the right-temple PPG signal are determined. A possibility of a cardiac dysfunction, a cerebral dysfunction, or both in the subject may be determined based on an EEG signal, a comparison of the pulse morphology data of pulses from the left-temple PPG signal and the right-temple PPG signal, or both.

### CROSS-REFERENCE TO RELATED APPLICATIONS

This application claims priority to U.S. Provisional Patent Application No. 63/050,000, filed Jul. 9, 2020, the disclosure of which is incorporated herein by reference in its entirety.

### FIELD

The present disclosure relates to system and apparatus for physiological and psychological parameter monitoring from a subject's head and methods of use thereof.

### BACKGROUND

Typically, wrist-based sleep monitors may rely on movement, heart rate and/or heart rate variability. They may detect sleep, but typically do not provide an accurate readout or measurement of the different sleep cycles. Hence, they are not useful for sleep assessment. Furthermore, devices that measure blood flow of the two cerebral hemispheres may be conducted with expensive and bulky functional near-infrared spectroscopy fNIRS equipment, which are also not suitable for sleep assessment. Hence, medical-grade sleep monitors are devices that are still typically used in a clinical environment, while consumer sleep monitor devices may be inaccurate for sleep assessment.

Further, a soldier may suffer mental and physical fatigue, potentially hindering operational readiness during deployment and reducing quality as active soldiers after suffering a traumatic event which may result in a potential long-lasting effect of brain deteriaration that may follow. Furthermore, with regard to the COVID-19 pandemic, a recent study on 725 COVID-19 patients has revealed that 15% of the hospitalized patients had acute neurological conditions. About ⅔ of the patients suffered from altered mental status while 1/3 of the patients suffered an acute ischemic stroke. Most of these patients had no prior conditions. Thus, for example, there is a need for continuous brain blood flow and activity monitoring to reduce a potential trauma.

### SUMMARY

In some embodiments, an exemplary method of the present disclosure may include continuously receiving, by a processor of a computing device, from a psychological and physiological sensing (PPS) device worn on a head of a subject, sensor data from a plurality of sensors fixed to the PPS device. The computing device may communicate with the PPS device. The plurality of sensors may include at least one left-temple photoplethysmography (PPG) sensor configured to be coupled to a left temple region of the head and at least one right-temple PPG sensor configured to be coupled to a right temple region of the head. The at least one left-temple PPG sensor may be configured to detect pulsating blood flow in blood vessels proximal to the left temple region and the at least one right-temple PPG sensor may be configured to detect pulsating blood flow in blood vessels to the right temple region. A left-temple PPG signal and a right-temple PPG signal may be continuously detected by the processor from the sensor data from the at least one left-temple PPG sensor and the at least one right-temple PPG sensor. At least one pulse morphology data of pulses related to the pulsating blood flow from the left-temple PPG signal and the right-temple PPG signal may be continuously determined by the processor. The least one pulse morphology data of each pulse in the left temple PPG signal and the right temple PPG signal may include at least one of a pulse amplitude of each pulse, a peak pulse amplitude of each pulse, or a rise time of each pulse. The at least one pulse morphology data of the pulses in the left temple PPG signal and the right temple PPG signal in a memory of the computing device may be stored by the processor. A possibility of a cardiac dysfunction, a cerebral dysfunction, or both in the subject may be determined by the processor based on a comparison of at least one of:
(i) at least one current pulse morphology data from the left temple PPG signal with at least one current pulse morphology data from the right temple PPG signal,
(ii) the at least one current pulse morphology data from the left temple PPG signal with at least one historical pulse morphology data from the left temple PPG signal stored in the memory, or
(iii) the at least one current pulse morphology data from the right temple PPG signal with at least one historical pulse morphology data from the right temple PPG signal stored in the memory.
   An alert of the possibility of the cardiac dysfunction, the cerebral dysfunction, or both, in the subject may be outputted by the processor on an output device of the computing device.

In some embodiments, an exemplary system of the present disclosure may include a computing device and a psychological and physiological sensing (PPS) device worn on a head of a subject comprising a plurality of sensors fixed to the PPS device. The plurality of sensors may include at least one left-temple photoplethysmography (PPG) sensor configured to be coupled to a left temple region of the head and at least one right-temple PPG sensor configured to be coupled to a right temple region of the head. The at least one left-temple PPG sensor may be configured to detect pulsating blood flow in blood vessels proximal to the left temple region and the at least one right-temple PPG sensor may be configured to detect pulsating blood flow in blood vessels to the right temple region. The computing device may include a memory, an output device, and a processor. The processor may be configured to execute software code stored in the memory that causes the processor to continuously receive sensor data from the plurality of sensors, where the computing device may communicate with the PPS device, continuously detect from the sensor data from the at least one left-temple PPG sensor and the at least one right-temple PPG sensor, a left-temple PPG signal and a right-temple PPG signal, continuously determine at least one pulse morphology data of pulses related to the pulsating blood flow from the left-temple PPG signal and the right-temple PPG signal, where the least one pulse morphology data of each pulse in the left temple PPG signal and the right temple PPG signal may include at least one of a pulse amplitude of each pulse, a peak pulse amplitude of each pulse, or a rise time of each pulse, store the at least one pulse morphology data of the pulses in the left temple PPG signal and the right temple PPG signal in the memory, determine a possibility of a cardiac dysfunction, a cerebral dysfunction, or both in the subject based on a comparison of at least one of:
(i) at least one current pulse morphology data from the left temple PPG signal with at least one current pulse morphology data from the right temple PPG signal,
(ii) the at least one current pulse morphology data from the left temple PPG signal with at least one historical pulse morphology data from the left temple PPG signal stored in the memory, or
(iii) the at least one current pulse morphology data from the right temple PPG signal with at least one historical pulse morphology data from the right temple PPG signal stored in the memory, and
   output an alert of the possibility of the cardiac dysfunction, the cerebral dysfunction, or both, in the subject on the output device.

### BRIEF DESCRIPTION OF DRAWINGS

Some embodiments of the disclosure are herein described, by way of example only, with reference to the accompanying drawings. With specific reference now to the drawings in detail, it is stressed that the embodiments shown are by way of example and for purposes of illustrative discussion of embodiments of the disclosure. In this regard, the description taken with the drawings makes apparent to those skilled in the art how embodiments of the disclosure may be practiced.

FIG. 1 schematically illustrates a psychological and physiological sensing (PPS) device worn on a head of a subject, in accordance with one or more embodiments of the present disclosure;

FIG. 2 schematically illustrates a system for monitoring at least one PPS device on at least one subject's head, in accordance with one or more embodiments of the present disclosure;

FIG. 3 illustrates graphs of an electrocardiogram measurement and dual photoplethysmogram (PPG) measurements, in accordance with one or more embodiments of the present disclosure;

FIG. 4 is a graph illustrating an exemplary pulse morphology of a pulse 365 detected in PPG measurements of pulsating blood flow in blood vessels proximal to a temple region in a head of a subject, in accordance with one or more embodiments of the present disclosure;

FIG. 5 illustrates a brain with cerebral arteries, in accordance with one or more embodiments of the present disclosure;

FIG. 6 is a signal flow diagram of sensor inputs in the PPS device, in accordance with one or more embodiments of the present disclosure;

FIG. 7 is flowchart of a first exemplary embodiment of a detected signal processing algorithm, in accordance with one or more embodiments of the present disclosure;

FIG. 8 is flowchart of a second exemplary embodiment of a detected signal processing algorithm, in accordance with one or more embodiments of the present disclosure; and

FIG. 9 is a flowchart of an exemplary method for physiological and psychological parameter monitoring from a subject's head, in accordance with one or more embodiments of the present disclosure.

### DETAILED DESCRIPTION

Among those benefits and improvements that have been disclosed, other objects and advantages of this disclosure will become apparent from the following description taken in conjunction with the accompanying figures. Detailed embodiments of the present disclosure are disclosed herein; however, it is to be understood that the disclosed embodiments are merely illustrative of the disclosure that may be embodied in various forms. In addition, each of the examples given regarding the various embodiments of the disclosure which are intended to be illustrative, and not restrictive.

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though it may. Furthermore, the phrases “in another embodiment” and “in some other embodiments” as used herein do not necessarily refer to a different embodiment, although it may. All embodiments of the disclosure are intended to be combinable without departing from the scope or spirit of the disclosure.

As used herein, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, terms such as “comprising” “including,” and “having” do not limit the scope of a specific claim to the materials or steps recited by the claim.

All prior patents, publications, and test methods referenced herein are incorporated by reference in their entireties.

In some embodiments, exemplary systems/apparatuses and methods of the present disclosure may be utilized to measure full cardiorespiratory activity in a sleeping subject that may enable an assessment of sleep disorders and determination of events like sleep apnea. When the blood flow is measured close to the brain, other disorders related to reduction in the blood flow such as occlusion of the carotid arteries. If we add a monitoring of the brain activity, other sleep disorders related to Parkinson's disease, or Post Traumatic Stress Disorder (PTSD) can be detected. These determinations may be used to better characterize the sleep disorders and to optimize intervention. They can also characterize the condition of a sedated patient in the ICU or anesthetized patient during operation.

In some embodiments, exemplary systems/apparatuses and methods of the present disclosure may be utilized to early detect and assess brain health deteriarations and their short term effect on cerebral blood oxygen supply and brain activity and long term effects on on sleep, cognitive and motor decline and PTSD, by using an affordable device, and in the comfort of one's own bed, which may be the key to early intervention, mitigation, and/or mass deployment. For example, exemplary systems/apparatuses and methods of the present disclosure may be utilized to perform such assessment in the field during or soon after a traumatic event so to prevent or minimize mental suffering and/or physical fatigue by soldiers, potentially hindering operational readiness during deployment and reducing their quality as active soldiers. For example, if such assessment is not conducted in the ICU or during an operation, a potential long-lasting effect of brain deteriaration may follow.

In some embodiments, exemplary systems/apparatuses and methods of the present disclosure may be utilized to offer an affordable and/or massively deployable medical monitor that can assess cognitive, emotional and cardio-respiratory ready a brain trauma or a danger deteriorations that would be crucial for improved intervention in cases where there is a brain trauma or a danger of brain trauma, in particular in the ICU, neurological and cardiac wards.

Furthermore, with regard to the COVID-19 pandemic, in some embodiments, exemplary systems/apparatuses and methods of the present disclosure may be utilized for an integrated physiological/cerebral monitoring of COVID-19 patients. As the carotid artery is often one of the first arteries to be clogged by plaque arising from cholesterol and/or due to blood coagulation, there is a need to monitor the physiological parameters related to blood flow and oxygen level near for example, monitoring by exemplary systems/apparatuses of the present disclosure may provide a more accurate brain health status taking into account potential blood flow problems between the heart and the brain.

In some embodiments, exemplary systems/apparatuses and methods of the present disclosure may be utilized to provide physiological and/or psychological disorder indications and/or, by measuring both modalities close to the brain, to be most accurate in determining potential brain damage. Exemplary disorders may include, but are not limited to, sleep disorders related to apnea and COPD, Alzheimer's and PTSD, cardiovascular events and/or pathological brain events related to stroke, anxiety, lack of attention (when needed). In some embodiments, exemplary systems/apparatuses and methods of the present disclosure may be utilized for subjects of all types, such as seniors and persons who may be at risk or during crucial military, civilian and medical missions. For example, in some embodiments, exemplary systems/apparatuses and methods of the present disclosure may be utilized for COVID-19 patients and other patients who may be anesthetized and ventilated prior to, during, and after hospitalization. For example, in the case of injury, exemplary systems/apparatuses and methods of the present disclosure may be utilized to assist in triage when there is a risk that the injury may lead to a lack of oxygenated blood being delivered to the brain and thus to a brain damage.

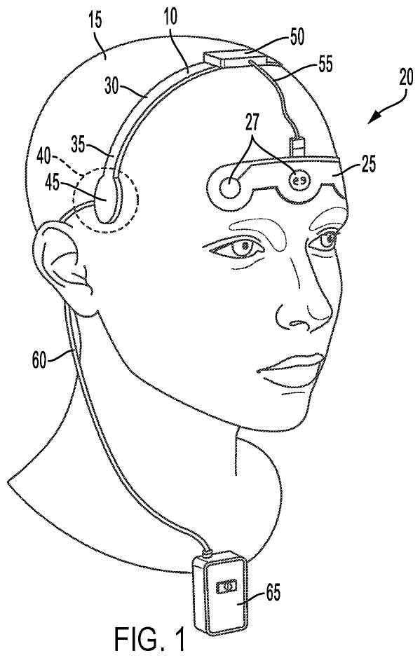

Embodiments of the present disclosure herein disclose a psychological and/or physiological sensing (PPS) device configured to be worn on a head (see FIG. 1) of a subject and methods of use thereof. The embodiments disclosed herein solve the above-stated problems in the background section of this disclosure in the development of a novel, medical grade device with a combination of EEG and physiological signal monitoring of a subject, which may be used in the clinic and/or in the field. The PPS device may continously monitor brain activity and cerebral blood/oxygen supply. The PPS device disclosed herein may further separate cerebrovascular-related and cardiorespiratory-related sleep disorders from psychological-related and emotional-related sleep disorders for improved diagnosis and optimal intervention.

The PPS apparatus may also be referred to herein as a PPS platform or a PPS device or a PPS head device. The PPS device may be configured to detect and measure neurological signals both from electrodes placed on the subject's forehead and/or electrodes placed over the temporal artery at the temples of the subject's head. Electro cardiac signals and/or temperature sensing signals and/or photoplethysmography (PPG) signals as well as other measurements based on light reflection of blood may be detected in output biosignal data and acquired from sensors and/or electrodes placed over the temporal artery.

The PPS device may assess differences in the output biosignal data from the biosensors placed on each side of the subject's head (i.e., over each temporal artery) and may use the differences to determine if a pathological state exists, such as whether the subject had a stroke, for example. The capability of measuring PPG pulse morphology from blood vessels in both hemispheres of the brain provides advantages over monitoring one side of the brain. For example, lower peak in a PPG signal measured on one side may suggest an arterial occlusion, and lower PPG peaks on both sides with respect to historical data may suggest a reduction in cardiac output which may be assessed through PPG pulse morphology comparison.

Furthermore, integrating blood flow and brain activity monitoring in the PPS device may enables a distinction between a dysfunction that has a neurological cause versus low blood/oxygen supply. For example, sleep that is disturbed by a reduced oxygen supply to the brain, in cases of congestive heart failure (CHF) or COPD, versus sleep that may be disturbed in subjects experiencing post-traumatic stress disorder (PTSD), for example. The PPS device disclosed herein is small and unobtrusive may be used by any healthcare professional. It may not require servicing for up to 12 hours.

In some embodiments, a controller unit of the PPS device may be configured to receive the raw (e.g., unprocessed) sensor signals from the various sensors and/or electrodes. The controller may be configured to process these sensor signals and to relay the raw sensor signals and/or the processed sensor signals over a communication network to a mobile device held on the subject's body and/or to a remote server over the communication network configured, for example, to receive the raw and/or processed sensor signals from a plurality of sensor devices connected on the PPS platform from a respective plurality of subjects located within a geographical region.

FIG. 1 schematically illustrates a psychological and physiological sensing (PPS) device 10 worn on a head 15 of a subject 20, in accordance with one or more embodiments of the present disclosure. PPS device 10 may include a multi-electrode patch 25 placed over and/or affixed to a forehead of subject 20. Multi-electrode patch 25 may include at least one electrode 27 affixed to the subject's forehead. A bridge 30 also known herein as a bridge member with a first end 35 and a second end (not shown, but right side of subject's head) may be configured to be placed over subject's head 15.

In some embodiments, the length of the bridge is configured such that the first end and the second end of the bridge may each include sensors 45 for measuring bioelectrical signals from the temporal arteries and placed and/or affixed in a temple region 40 of the subject's temples from electrodes 27 along the subject's forehead and/or from the sensors 45 from the left and right temporal arteries respectively from the left and right side of subject's head 15.

In some embodiments, PPS device 10 may further include a cable 55 that connects electrodes 27 in multi-electrode patch 25 to an electronic circuitry housing 50 which may include circuitry. Cable 55 may be coupled to electronic circuitry housing 50 with a snap connector, for example, or any other suitable connector.

In some embodiments, bridge 30 may include a lumen which may allow electrical wires or shielded conductors to be placed therein to connect circuitry in electronic circuitry housing 50 to sensors 45. A cable 60 may connect the circuitry in electronic circuitry housing 50 and/or signals from sensors 45 to a power unit 65.

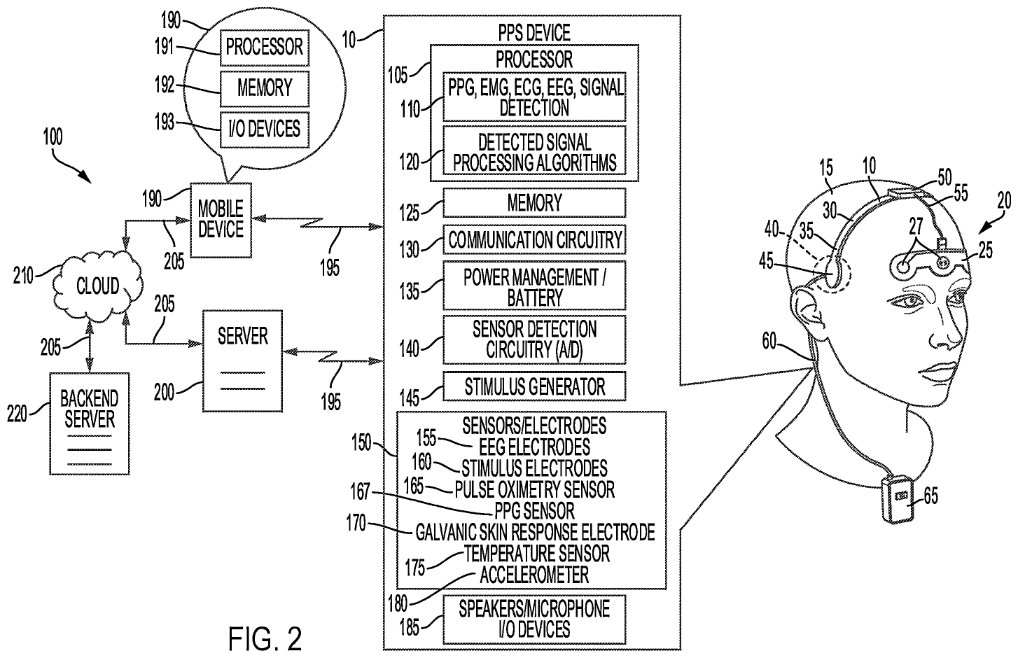

FIG. 2 schematically illustrates a system 100 for monitoring at least one PPS device 10 on at least one subject's head 15, in accordance with one or more embodiments of the present disclosure. FIG. 2 illustrating one PPS device 10 on head 15 of one subject 20, is merely for conceptual clarity and not be way of limitation of the embodiments disclosed herein. System 100 may be used to monitor any number of PPS devices 10 for any respective number of subjects 20.

System 10 may include at least one PPS device 10 as shown in FIG. 2 communicating 195 with a mobile computing device 190 and/or a server 200. The mobile computing device 190 and/or the server 200 may communicate 205 over a communication network 210 such a cloud computing communication network to a backend server 220. In some scenarios, the backend server 220 may be used to monitor the at least one PPS device 10 on at least one soldier in a combat environment. In another scenario, backend server 220 may be used to monitor the at least one PPS device 10 on at least one patient to detect cardiovascular/cardiorespiratory and/or pathological brain events as a result of suffering or having suffered from the COVID-19 exposure, sepsis and related sicknesses leading to acute respiratory distress syndrome (ARDS) or to other reasons which may require anesthesia and ventilation.

In some embodiments, as shown in the block diagram of FIG. 2, PPS device 10 may include a processor 105, a memory 125, communication circuitry 130, power management circuitry 135 including a battery, sensor detection circuitry 140 including analog-to-digital converters (A/D), a stimulus generator 145, sensors and/or electrodes 150, and input and/or output devices (I/O) such as a speaker and/or microphone for the subject to hear audio signals relayed over the communication network and/or to transmit the subject's voice over the communication network.

In some embodiments, communication circuitry 130 may include Bluetooth circuitry, Wi-Fi circuitry, cellular circuitry and/or any other suitable wireless and/or wired communication circuitry of any suitable protocol and/or standard, for communicating 195 directly with mobile computing device 190, server 200, and/or for communicating 205 with backend server 220 over communication network 210 using any suitable communication protocol. Communication circuitry 130 may include at least one antenna and/or any suitable communication interface.

In some embodiments, sensors and/or electrodes 150 may include, for example, any number of EEG electrodes 155, stimulus electrodes 160 for applying a stimulus to subject's head 15 from stimulus generator 145, pulse oximetry sensors 165, photoplethysmography (PPG) sensors 167, galvanic skin response electrodes (ECG) 170, temperature sensors 175, accelerometer 180 and/or other optical transmitters and receivers for measuring different reflections from white or red blood cells or from other particles in the blood, which may indicate the amount of oxygen saturation, the amount of white blood cells, the amount of flow, coagulation and/or other blood related measures such as cholesterol. Note that PPG sensors 167 may include pulse oximetry measurement capabilities. In other embodiments, other sensors such as those for analyzing sweat may be attached at the temporal lobe to examine the level of oxytocin and other measurements that may be obtained from sweat.

Processor 105 may be configured to execute software code stored in memory 125 such as a signal detection module 110 for detecting signals detected and/or generated from sensors and electrodes 150 and for distinguishing in those signals, photoplethysmography (PPG) signals, electromyography (EMG) signals, electrocardiogram (ECG or EKG) signals, and/or electroencephalogram (EEG) signal in the subject's body. Detected signal processing algorithms 120 may be used to compute and/or correlate any suitable parameter and/or metric associated with the detected signals in accordance with the embodiments described herein.

In some embodiments, a three electrode (e.g., electrodes 27) multi-sensor patch 25 may be used for sensing and/or stimulating brain activity. In other embodiments, electrodes on the multi-sensor patch 25 on the forehead may be connected to electronic circuitry for recording a EEG-data. Full cognitive/emotional and sleep hypnograms may be obtained from multi-sensor patch 25 with electrodes 27. Note that the embodiments shown here are merely for conceptual and/or visual clarity, and not by way of limitation of the embodiments disclosed herein. Any number of electrodes, patches, and/or sensors may be placed on the subject's head and/or body to be used for performing the functions described herein.

In some embodiments, bridge 30 over the skull may support and/or position the two sensing members on the two (left/right) temporal arteries (e.g., sensors 45). Sensors 45 placed at each temporal artery may include PPG sensors 167, temperature sensor 175 and/or galvanic skin response electrode (ECG) 170, so that the cumulative detected signals from the sensors may accurately measure heart electrical activity. PPG sensors 167 at both hemispheres may provide an early indication of blood flow problems to either brain hemisphere. In that case, blood flow and/or pulse oximetry distributions may be stored in memory for different heart rates. Then, if the current reading of the heart rate, the blood flow, and/or pulse oximetry is above or below a preset number of standard deviations from the center of the distribution, (e.g., for a given heart rate, or for a given combination of any parameters), an alert may be produced. Alerts may be produced when the difference in any of the parameters as measured in either temporal lobe is above a certain preset number of standard deviations (e.g., a predefined threshold) from the center of the distribution of that difference. Combined, these two pulse oximeters may detect sleep apnea, stroke, cardiac disorder related to blood flow, to heart rate or heart rate variability. On the two temporals, galvanic skin response electrodes 170 may be used to sense the heart electrical activity.

In some embodiments, sensors 45 may include three sensors at the temporals: PPG sensor 167 with pulse flow morphology, temperature sensor 175, and electro dermal activity and galvanic skin response electrode 170. Galvanic skin response electrode 170 may be a conductive electrode for sensing and stimulating the brain. It may also be used to sense the cardiac QRS complex using the difference from the electrode on the other temporal. Redundancy in sensors 45 may improve reliability of the measurements, particularly under field conditions. Moreover, sensor redundancy will enable a comparison between pulse morphology and pulse oximetry level at each temporal artery. This is important for detection in the subject of potential interruptions in blood flow to one side of the brain, for example, in case of a stroke or a bleeding wound.

In some embodiments, processor 105 (e.g., algorithms 120) may correlate (in time) the heart electrical activity with the pulse oximeter measurements, so as to provide an indication of blood velocity flow which is a proxy to blood pressure. The heart rate and heart rate variability (HRV) may be calculated to provide a further indication of cardiac health, sympathetic and parasympathetic activity. In addition, together with the EEG sensor and the sleep segmentation, the stage of the sleep cycle an individual experiences sleep apnea may be assessed as well as other events such as heart rate decelerations or blood flow dips may occur, thus providing a full picture of the whole sleep process. Temperature sensors 175 at each side of the temples may assist in the detection of an inflammation developing in the body, in that the personalized base line temperature of each subject may be recorded, even during different sleep cycles. It may be possible that measuring body temperature may affect the air-conditioning system in the house, for sleeping at an optimal temperature if detected by the sleep analysis. It may be possible that certain music and/or other stimulation may be applied to improve the sleep, for example, such as reaching a deep sleep state faster and staying in that state longer.

In some embodiments, electronic circuitry housing 50 may include electronic circuitry that serves as a front end to the sensors 45, including sensor detection circuitry 140 with an ultrasensitive analog-to-digital (A/D) converter and a microcontroller unit (MCU) including processor 105 to send data to communication circuitry 130 such as a Bluetooth, a WiFi, an optical and/or acoustic transmitter, for example. Sensor detection circuitry 140 may also include a smaller and low frequency Analog-to-Digital Converter (A/D) to sample the electrical dermal activity and/or the temperature at the temporals in sensors 45. The pulse wave of the pulse oximeter may be sampled for more advanced analysis beyond pulse oximetry values. Electronic circuitry housing 50 may include an accelerometer and gyro configured to detect concussions, body position, falls, movement and step counts as well as cardio ballistic signals during sleep for estimation of cardiac flow and muscle contraction.

In some embodiments, power unit 65 may include an accessory MCU, battery 135 and transmission device 130 (e.g., communication circuitry). PPS device 10 may include electrical circuitry configured to minimize power consumption during continuous modes of operation.

In some embodiments, a PPG/ECG combination sensing module for sensors 45 may be provided by Maxim Integrated Part No. MAX86150EFF. An integrated pulse oximetry and heart rate monitoring system may be provided by Maxim Part No. MAX30101. (Maxim Integrated, San Jose, CA, USA). A temperature and humidity sensor may be provided by Microchip Technology Inc. Part No. MCP9700-E/TO (Microchip Technology Inc., 2355 West Chandler Blvd. Chandler, Arizona, USA). A 9-axis gyroscope and accelerometer may be provided by INVENSENSE MPU-9250 9-AXIS SENSOR (InvenSense, Inc. San Jose, CA USA). A galvanic skin sensor may be provided by Seeed Studio Grove GSR Sensor Module 3.3V/5V Open Source, Galvanic Skin Response Sensor (Seeed Studio Electronics, Shenzhen, China).

In some embodiments, processor 105 may measure the time difference in the peak PPG pulses from the PPG sensor and peaks in the ECG signal (e.g., the QRS complex) in the heart electrical activity for providing an indication of blood flow velocity and proxy of blood pressure. This measurement may be used to determine if during sleep, the subject may experience a sudden reduction in blood pressure or blood oxygenation, thus identifying a cardio/respiratory medical problem which may lead to damage to internal organs and/or the brain. Furthermore, measuring temperature during sleep may provide an indication of a gland's overactivation producing sweat. Sweat may be further correlated to sleep stages to indicate heart rate variability (HRV) and activation of the sympathetic nervous system, which may indicate traumatic episodes of the subject during sleep. Electronic circuitry housing 50 may also include an ambient temperature sensor (pointing away from the body) to analyze the temperature difference between the body and the environment. This may help to more accurately determine whether the glands and/or other organs may be affecting the body temperature and to separate that from the effect of the environment.

In some embodiments, correlation data may be obtained between the environmental temperature and the body temperature, so that deviations from the correlation (in standard deviations) may be recorded and used for generating alerts.

In some embodiments, PPS device 10 may include multi-electrode patch 25 as a disposable forehead sensor with a snap connector for attachment to the electronic circuitry housing 50.

In some embodiments, PPS device 10 may include electronic circuitry in electronic circuitry housing 50 positioned at the central portion of head 15 so as to reduce mechanical weight constraints of PPS device 10. This may enable the subject to wear PPS device 10 for longer periods of time so as to improve the accuracy of the measurements and to be able to detect events right when they occur. Sensors 45 may include an adhesive that can stay on the skin for extended periods of time under different conditions including military field conditions, resilient to dust and sweat. The EEG electrodes 27 may include a dry gel for optimal contact with the skin and may adhere for prolonged periods of time. The gel may be designed to reduce skin irritation and to enable the skin to breath.

In some embodiments, the shape of the sensor, and identification of the appropriate gel and electrodes contacting the gel may be optimized. Improved electrical conductance may be involved to investigate the signal from the brain, accompanied by associated biomarker results as well as measurements of the signal-to-noise ratio and frequency response. For example, measuring a higher frequency response may demonstrate that the electrodes are more sensitive. PPS device 10 may include electrodes that are comfortable in that they do not move from the placement site on the subject's heads and maintain 12 hours of conductance for measuring the physiological signal.

In some embodiments, PPS device 10 may include a six or more axes accelerometer with automatic alerts for concussion and head injuries. The accelerometer 180 may be used to acquire data for observing head movement during sleep. Processor 105 executing detected signal processing algorithms 120 software module may use the accelerometer data for removing artifacts from the detected and/or acquired physiological and EEG data.

In some embodiments, by placing electronic circuitry housing 50 on the subject's head with a small-sized printed circuit board may improve accelerometer measurement sensitivity. This improved sensitivity may enable observation of small movements by the head and quantified such as measurements of cardio-ballistic movements. In some embodiments, detected signal processing algorithms 120 software module may apply filtering algorithms to correct for drift and/or events in the cardio-ballistic signal.

In some embodiments, the PPS device 10 may be used to monitor elderly adults that are prone to blood flow disruptions due to cardio/vascular/respiratory problems. The PPS device 10 may detect disruption in oxygenated blood to the brain and may alert the elderly adult to sit down, before a potential (momentary) blackout occurs which may lead the subject to fall.

In some embodiments, the mechanical design of the bridge 30, which may be also referred to as a temporal bridge, positioned between two temples is such that the weight of the electronics in electronic circuitry housing 50 may be supported by an adhesive patch on the forehead. The mechanical forces reduce the attachment of the patch to the skin. In other embodiments, the bridge 30 design may be optimized to incorporate these mechanical forces so as to keep the electrode patch better attached. The bridge 30 may be formed from a thin flexible material so that long periods of placement will not cause discomfort to the subject. The bridge 30 may be designed for different head sizes. The bridge 30 may be flexible to accommodate different head contours that enables the sensors and electrodes to produce satisfactory responses without interfering with the subject's sleep.

In some embodiments, the bridge 30 may include a first end and a second end with a predefined length from the first end to the second end, such that the sensor 45 fixed to the first end may detect biosignals from the left temple of the subject and sensors 45 fixed to the second may detect biosignals from the right temple. The predefined length of the bridge may be adjusted to ensure that the sensors fixed to the first end and the second end are positioned to contact the left temple and the right temple, respectively.

In some embodiments, the electronic circuitry of PPS device 10 may be configured to operate in two modes: a field mode and an extensive mode. The extensive mode may relay data to cloud backend server 220 via cloud communication network 210 in real-time and cloud backend server 220 may perform deeper signal analysis on the relayed. The field mode may include PPS device 10 performing a limited signal analysis to enable data readout via Bluetooth to nearby computing device 190 such as a tablet, or to cause the illumination of a red LED light (not shown) on PPS device 10 to indicate an emergency alert. Such an emergency alert may indicate a lack of oxygenated blood to the brain. In the case of soldiers, it may be helpful for triage.

In some embodiments, PPS device 10 may provide real time physiologic monitoring of blood flow with oxygen level, as well as heart rate and heart rate variability (HRV). The physiological sensing signals may be measured at the level of the brain, so as to eliminate the possibility of a carotid blockage as a pathological event affecting the subject. In other embodiments, PPS device 10 may further measure activity from both sides of the brain to identify a blockage in one hemisphere of the brain. Thus, PPS device 10 may be used to determine if sleep disorders are related to cardiorespiratory malfunction. If the subject, for example, suffers from disordered sleep states, elimination of cardiorespiratory factors may assist in PTSD identification and differentiation from sleep apnea.

In some embodiments, PPS device 10 may be used as a comprehensive bioanalytical tool for sleep monitoring that may provide data on psychological and cardiovascular sleep disorders.

In some embodiments, at least one PPS device 10 may be used as a comprehensive brain/body monitoring for a plurality of special forces soldiers or others in critical missions, for example, that may provide real-time alerts on level of alertness and executive functioning of soldiers, e.g. soldiers' physical or mental stress level, and whether cardiac activity is uncontrollable. The at least one PPS device 10 may be managed by backend server 220, for example, located in a military command and control center. In case of a wounded soldier in the field, the at least one PPS device 10 may alert to blood flow and oxygen level deficiencies. Most importantly, the at least one PPS device 10 may provide indications on development of brain injury resulting from the lack of oxygenated blood to the brain.

In some embodiments, speaker and/or microphones 185 coupled to the at least one PPS device may be used to communicate with at least one subject wearing the at least one PPS device 10. The speakers may be in-ear speakers, or may be bone-conducting speakers placed on the bone next to the ear extended from sensors 45 at temple region 40.

In some embodiments, the at least one PPS device 10 may provide early alerts in the development of age-related diseases for seniors, such as congestive heart failure and chronic obstructive pulmonary disease (COPD). It may also indicate brain disorders related to early neurodegeneration, or to post trauma, by providing a comprehensive picture of health at a fraction of the cost of hospital-based monitors with minimal size, portability, and easy administration at the clinic or at home by the subject.

In some embodiments, the ability to interpret brain activity in real-time together with the ability to interact with the subject verbally and through music, for example via Alexa from Amazon (Cortana, Siri, or any other interactive speech enabled tool) enables the creation of a virtual care giver (VCG). The VCG may monitor the subject and respond to changes in brain activity. The VCG caregiver may respond, for example, when PPS device 10 may detect that cognitive activity is lower than the daily average, when anxiety is higher than the anxiety around the same time of day on other days, and/or when sleep is different than in other days (either better or worse).

In some embodiments, when integrating physiological measurement using PPS device 10, the VCG may respond to reduced physical activity, such as walking less steps during the day, a slower time to get to the bathroom than normal, and/or waking up at night more time than usual, for example. The VCG may respond to changes in heart rate, HRV, blood flow velocity, measurement differences in pulse oximetry between the two temporal arteries, changes in temperature in comparison to the normal temperature at that time of day, changes in temperature between the two temporals, changes in electrodermal activity, and/or changes in any of the other biomarkers described above.

In some embodiments, the response of the virtual caregiver may be to encourage the subject to get out of bed if the subject is staying in bed longer than usual, or to go to sleep earlier if the previous night's sleep was detected to be a poor quality of sleep. The VCG may initiate playing relaxing music to the subject through the speakers if anxiety is detected, or to perform a cognitive or emotional automatic assessment to obtain a clear indication of the status of the subject. It may suggest a more cognitively challenging movie to watch, or a lecture to listen to, if a reduction in cognitive activity is observed. In other embodiments, the VCG may alert family members to a pathological state of the subject being monitoring. It may initiate a video/phone call between the participant and a family member or a friend who often improves the mood of the participant when needed. In other scenarios, the VCG may alert a physician or medical alert teams if the medical state of the subject appears to be more urgent or life-threatening, for example in the case of a hard fall detected by the accelerometer on the subject's head, or when blood flow measurements are detected below a pre-determined threshold.

In some embodiments, sensors 45 on the temporal arteries may be extended to provide a bone conductance speaker on both sides, and/or sensors 45 equipped with a microphone for a full interactive system, potentially for the speech or hearing-impaired. Virtual and/or augmented reality goggles may be added to the system for entertainment, brain training or socialization.

In some embodiments, detected signal processing algorithm module 120 may represent each of the different physiological and/or psychological measurements xᵢ(t), i∈1 . . . N as a time series of measurements sampled at a different frequencies. By interpolating each measurement, all measurements to be sampled at the same (higher) frequency may be obtained. Thus, time series vector may include having all measurements at the same frequency. Let eⱼ(t), j=1 . . . K be a vector, denoted E (t) of environmental factors such as temperature, time of day, and other external parameters to be found useful, like the day of the week. Now, for each specific measurement, xᵢ(t), the conditional distribution xᵢ(t)|E(t) may be estimated. E(t) may be discretized so that the distribution of xᵢ(t) may belong to a finite collection of distributions for each E. In some embodiments, the conditional distribution may be further approximated as a Normal or Poisson distribution with a standard deviation σᵢ(E).

In some embodiments, processor 105 may generate an alert when a specific measurement xᵢ(t) falls outside the mean of the distribution by a preset number of standard deviations λᵢ(E) and/or for a preset minimal time duration. In other embodiments, processor 105 may generate an alert if a vector of measurements xᵢ(t), i∈S falls outside of the corresponding distribution or cluster center.

In some embodiments, detected signal processing algorithm module 120 may use general machine learning algorithms to predict different measurements xᵢ using the other measurements xⱼ and/or the environmental parameters E. Processor 105 may generate a distribution of the error between the predicted value and the actual value, and keep only those predictions that have a small error distribution. Processor 105 may generate alerts when the actual measurements falls outside of the error distribution as above.

The embodiments shown in FIG. 2 are merely for visual and conceptual clarity, and not by way of limitation of the embodiments disclosed herein. For example. the PPS device 10 may not include processing capabilities (e.g., processor 105), but may include the EEG electrodes 155 positioned on a forehead of the subject 20 along the ECG sensors 170 and/or the PPG sensors 167 coupled to each of the temples on the head of the subject. The detected sensor data may be processed by the sensor detection circuitry 140 and converted to sensor signals. In other embodiments, the sensor signals may be communicated 195, or transmitted by the communication circuitry 130 for further processing by any of the mobile computing device 190, the server 200, and/or the backend server 220. The mobile computing device 190, for example, may include a processor 191, a memory 192, and/or input/output (I/O) devices 193 where the processor 191 may be capable of executing the signal detection module 110 and/or the detected signal processing algorithms 120 of processor 105. The server and/or the backend server 220 may similarly include a processor, a memory, and/or input/output (I/O) devices for performing the detected signal processing algorithms 120 of processor 105.

In some embodiments, the detected signal processing algorithm module 120 may use the measurements from both sides of the temporal artery separately, and processor 105 may generate alerts when there is a large difference, based on a preset deviation from the center of the distribution of the difference, between the two-temporal measurements, as described above.

In some embodiments, the PPS device 10 may be used for dual photoplethysmography (PPG) monitoring provides early indications of arterial occlusion and detection of a possibility of a cardiac dysfunction, a cerebral dysfunction, or both in the subject. In this case, the PPG sensors may be placed in the right and left temple regions 40 as shown in FIGS. 1 and 2. The cerebral dysfunction may include a detection of a hemorrhagic stroke or an ischemic stroke.

In some embodiments, the PPS device 10 may be used as a physiological/cerebral monitor in the Intensive Care Unit (ICU) or during operation to monitor the depth of anesthesia via the EEG sensor 155, to monitor the blood flow and oxygenated blood supply to the brain, under anesthesia or under sedation in the case of ventilated patients such as in sepsis or COVID-19. The EEG sensor 155 may monitor the brain activity under sedation and during sedation interrupts and may indicate the level of agitation and pain of the patient and indicate abnormal electrical activity such as pre-ictal, ictal and burst suppressions. Together, this may provide a comprehensive monitor that is needed in the ICU and during operations.

There are approximately 400 miles of blood vessels in the brain, which may consume more than 20% of the total oxygen supply. Thus, low blood flow, resulting from cardiac dysfunction or blood coagulation, may lead to occlusion of thin blood vessels as well as major arteries. This may cause brain damage, which may become irreversible within a few hours. A comparison of the morphology of the PPG between the two hemispheres may indicate changes that are due to coagulation or other interruptions of blood supply.

Furthermore, the pulse morphology of the PPG pulses may carry additional information and may be correlated with physiological and functional changes in the cardiovascular system. The embodiments herein leverage these parameters such that the PPS device 10 may be used to measure the right and left temple PPG signals and use the bilateral symmetry or asymmetry in the PPG signals from both temples to detect micro, mini and major arterial occlusions early associated with the brain and/or cardiac dysfunction, so as to enable timely intervention for improving patient outcomes.

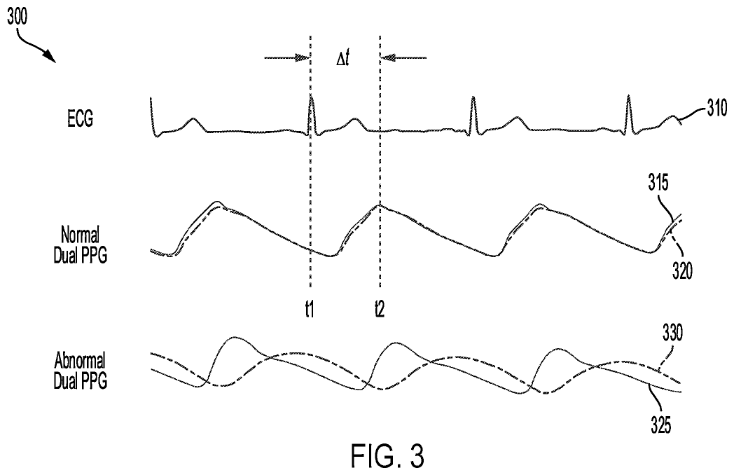

FIG. 3 illustrates graphs 300 of an electrocardiogram (ECG) measurement and dual photoplethysmogram (PPG) measurements, in accordance with one or more embodiments of the present disclosure. The graphs 300 illustrate an ECG measurement 310, a normal Dual PPG measurement with a PPG measurement 315 detected by a PPG sensor placed at the left temple and a PPG measurement 320 detected by a PPG sensor placed at the right temple, and an Abnormal Dual PPG measurement with a PPG measurement 325 detected by a PPG sensor placed at the left temple and a PPG measurement 330 detected by a PPG sensor placed at the right temple. The normal dual PPG signals 315 and 320 nearly overlap with one another exhibiting bilateral symmetry. This indicates that the pulsed blood flow to the two hemispheres of the brain is in-phase indicative of a healthy brain.

However, if there is an occlusion or blood pressure changes in one of the arteries in one hemisphere of the brain that typically occurs when there is an arterial blockage or during a stroke, for example, then blood vessels feeding one hemisphere of the brain may have a blood volume lower than the other hemisphere which may be indicative that the subject may have had a stroke. These effects may cause an asymmetry in a train of PPG pulses detected in the PPS measurements at the right and left hemispheres as shown in PPG measurements 325 and 330. Hence, a comparison of the pulse morphology of the PPG signals in the temporal arteries of the right and left hemisphere may be used to determine occlusions and impaired oxygen supply in one or both hemispheres, but a stroke typically affects one hemisphere. For example, if there was an occlusion in the blood vessels in the right hemisphere due to an ischemic stroke, the blood flow would be lower in the right hemisphere so the right PPG measurement would be indicative of the PPG measurements 330 shown in FIG. 3 and the left PPG measurement indicative of the PPG measurements 325 as shown in FIG. 3.

The ECG measurement 310 shows timestamp marker t1 at a time where the heart electrically generates series of QRS complexes detected in the ECG signal measured by the ECG sensor 170 also placed at the right and/or left temple of the subject's head. Thus, as the heart pumps blood through body, the delay Δt between a measured PPG peaks occurring at a timestamp t2 on the right and left symmetric PPS graphs and the timestamp marker t1 of the QRS complex may be used to compute the blood flow velocity in the blood vessels in the brain proximal to the right and left temples of the subject's head.

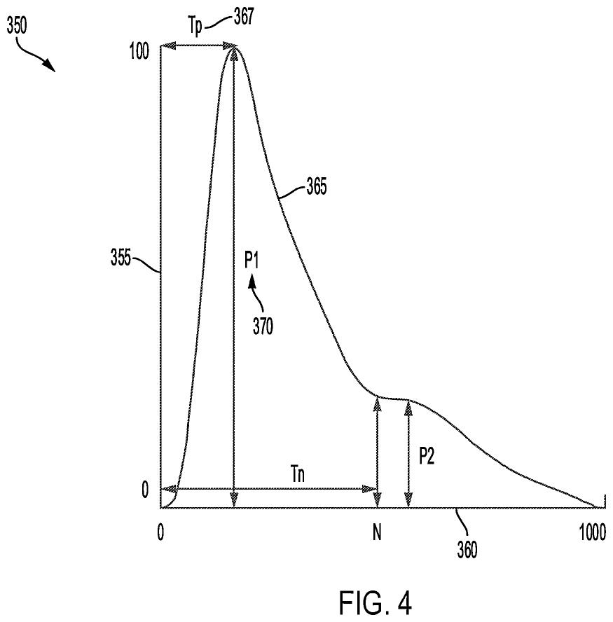

FIG. 4 is a graph 350 illustrating an exemplary pulse morphology of a PPG pulse 365 detected in PPG measurements of pulsating blood flow in blood vessels proximal to a temple region in a head of a subject, in accordance with one or more embodiments of the present disclosure. The graph 350 is shown with a time x-axis 360 and a signal amplitude on the Y-axis 355. FIG. 4 depicts the key parameters for characterizing pulse morphology also known herein as pulse morphology data. The PPG pulse 365 may be characterized, at least in part, by the following parameters: a peak PPG pulse amplitude 370 denoted as P1 and a PPG pulse rise time 376 denoted as Tp of the pulse 365. A peak PPG pulse amplitude of pulse in a left-temple PPG signal may be denoted herein as P1L, and P1R for a right-temple PPG signal.

In some embodiments, the PPS device 10 may be used to detect a respiratory deficiency in the subject 20 when the processor detects no change in the PPG pulse morphology between the PPG measurement 325 detected by the left-temple PPG sensor and the PPG measurement 330 detected by the right-temple PPG sensor, and yet both the left-temple PPG sensor and the right-temple PPG sensor both detect that a level of blood oxygen saturation (SpO2) on both sides of the brain has been reduced.

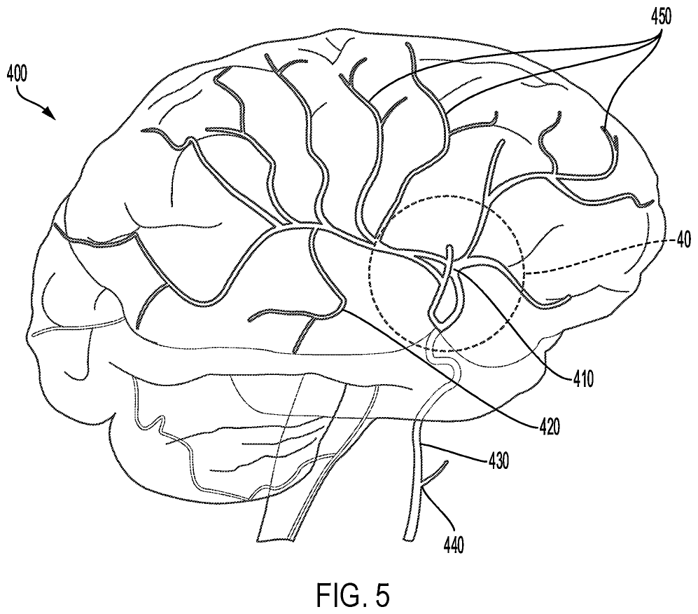

FIG. 5 illustrates a brain 400 with cerebral arteries, in accordance with one or more embodiments of the present disclosure. The brain 400 may include an anterior temporal artery 410, a middle temporal artery 420, an internal carotid artery 430 and an external carotid artery 440. The temple region 40 in the temple of the head may include the middle cerebral artery (MCA) that is sampled by the left-temple and right-temple PPG sensors. The MCA is the largest branch of the internal carotid which supplies blood to the frontal lobe and the lateral surface of the temporal and parietal lobes, including the primary motor and sensory areas of the face. The MCA is the artery most often occluded in a stroke. Thus, placing the left-temple and right-temple PPG sensors over the region 40 in which the MCA is located, may be optimal for a detection of occlusion and stroke. Moreover, comparing PPG morphology parameters associated with specific physiological features and morphology deviations between the two temporal arteries may indicate specific one-sided physiological changes. Thus, a continuous dual-PPG morphology monitor, such as PPS device 10, may assist in earlier detection of dysfunctions in cerebral blood flow and oxygen supply as described in the flowcharts of the following figures.

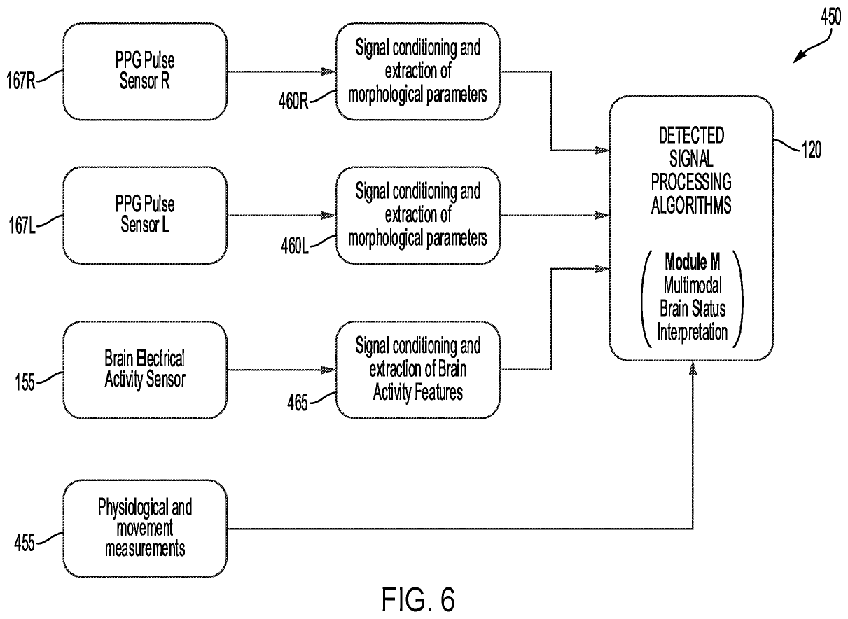

FIG. 6 is a signal flow diagram 450 of sensor inputs in the PPS device 10, in accordance with one or more embodiments of the present disclosure. The PPG pulse sensor 167R may be placed over the right temple of the subject 20 and the sensor data may undergo a signal conditioning and extraction of morphological parameters 460R implemented by the sensor detection circuitry 140 and the signal detection block 110. The PPG pulse sensor 167L may be placed over the left temple of the subject 20 and the sensor data may undergo a signal conditioning and extraction of morphological parameters 460L implemented by the sensor detection circuitry 140 and the signal detection block 110. In other embodiments, a brain electrical activity sensor 155 implemented, for example, by at least one EEG sensor on multi-electrode patch 25 may be placed on the forehead of the subject 20. The sensor data from the brain electrical activity sensor 155 may undergo a signal conditioning and extraction of brain activity features 465 implemented by the sensor detection circuitry 140 and the signal detection block 110.

In some embodiments, the processed PPG signals and brain activity features may be relayed to the detected signal processing algorithms module 120. Similarly, additional sensor measurements from the ECG sensor 170 and/or from the accelerometer 180 for detecting physiological and/or movement measurements 455 may be relayed to the detected signal processing algorithms module 120.

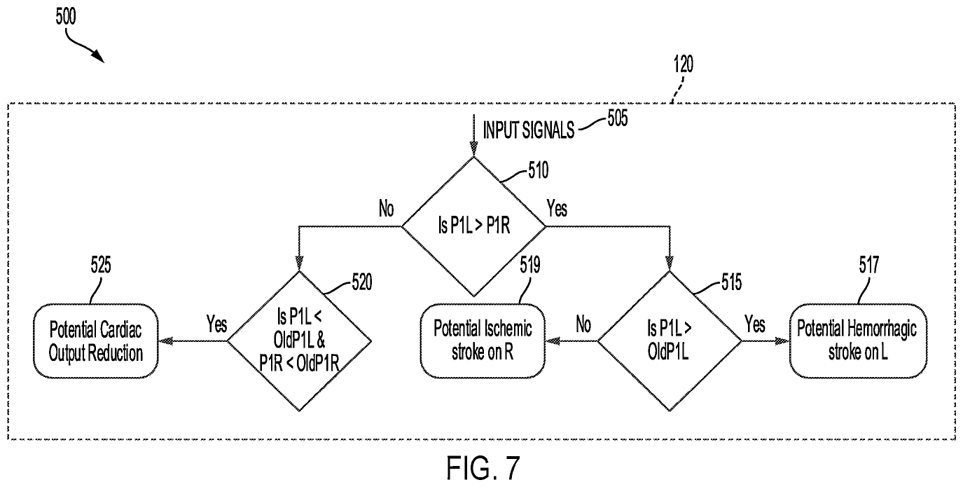

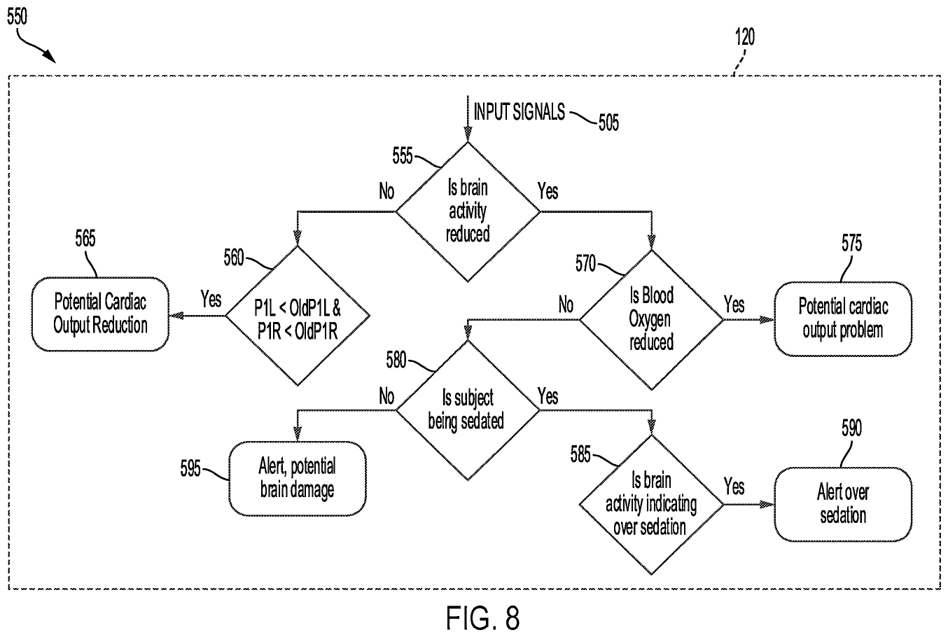

In some embodiments, the detected signal processing algorithms module 120 may be configured to use the processed output sensor data to provide information about a possibility of cardiac dysfunction (e.g., cardiac output reduction), a cerebral dysfunction (e.g., hemorrhagic stroke, ischemic stroke), brain damage due to reduction in blood oxygen, and/or over sedation alerts when the subject 20 may be over-sedated. In FIGS. 7 and 8, the detected signal processing algorithms module 120 may be executed by any processor of the following computing devices: the processor 105 incorporated into the PPS device 10, the processor 191 from the mobile computing device 190, and/or the processor associated with the server 205 and/or the backend server 220.

FIG. 7 is flowchart 500 of a first exemplary embodiment of the detected signal processing algorithm 120, in accordance with one or more embodiments of the present disclosure. The processing of data from a PPG Pulse Sensor 167R (e.g., from the PPG sensor placed over the right temple) and the data from a PPG Pulse Sensor Data 167L (e.g., from the PPG sensor placed over the left temple) may be used to detect a right-temple PPG signal and a left-temple PPG signal as shown in the Normal Dual PPG signals 315 and 320 of FIG. 3.

In some embodiments, the processor (e.g., the processor 105) executing the software modules: the signal detection 110 module and the detected signal algorithm 120 module may then determine from the right-temple PPG signal and the left-temple PPG signal (e.g., the PPG signals 315 and 320 in FIG. 3), at least one pulse morphology data of pulses related to the pulsating blood flow may be extracted from the left-temple PPG signal and the right-temple PPG signal as shown in FIG. 4. For example, the at least one pulse morphology data may include peak pulse amplitudes P1L and P1R respectively extracted from the left-temple PPG signal and the right-temple PPG signal and timestamps such as t2 of FIG. 3 of the PPG pulse peaks.

In some embodiments, this data may be stored in the memory 125, for example. Data acquisition of the right and left temple PPG signals may be acquired over any suitable predefined period of time and stored in the memory as historical data. The historical data may be recalled or fetched by the processor from the memory at any time (e.g., OldP1L and OldP1R) and compared to the new or currently-acquired data (P1L and P1R) from the right and left temple PPG signals for determining cardiac and/or cerebral dysfunctions.

In some embodiments, the detected signal algorithm 120 module may extract signal data from the right and left temple PPG signals as the input signals 505 to the flowchart 500. The processor may first determine in a decision step 510 if P1L>P1R. If so, the processor may then determine in a decision step 515 if P1L>OldP1L. If so, this would be indicative of a potential hemorrhagic stroke of the left side of the brain 517 where there is more blood in the brain arteries of the left hemisphere as previously measured due, for example, from a ruptured blood vessel in the brain. If not, then this may be indicative of a potential ischemic stroke on the right side of the brain where there may be less blood in the arterial system of the right hemisphere of the brain compared to the left hemisphere due to occlusions in the right hemisphere arterial system. This scenario for a potential ischemic stroke may be shown for the Abnormal Dual PPG signals of FIG. 3, for example as previously described.

In some embodiments, if in the decision step 510, P1L is not greater than P1R, the processor may evaluate if P1L<OldP1L and P1R<OldP1R in a decision step 520. If so, the processor may assess that there is a potential cardiac output reduction 525.

FIG. 8 is flowchart 550 of a second exemplary embodiment of the detected signal processing algorithm 120, in accordance with one or more embodiments of the present disclosure. This flowchart may be used to assess a possibility of cardiac dysfunction (e.g., cardiac output reduction), brain damage due to reduction in blood oxygen, and/or over sedation when the subject 20 may be over-sedated if the subject 20 is receiving medication for sedation. The detected signal algorithm 120 module may extract signal data from the right and left temple PPG signals as well as the brain activity features from the brain electrical activity sensor 155 and provided as the input signals 505 to the flowchart 550.

In some embodiments, in a decision step 555, the processor may assess if the brain activity is reduced by measuring a decrease in the extracted brain activity features. If not, the processor may further assess in a decision step 560 whether P1L<OldP1L and P1R<OldP1R. If so, the processor assesses that the subject 20 may have a potential cardiac output reduction 565. If not, the processor assesses from the PPG data if there is a reduction in blood oxygen in a decision step 570. If so, the processor assesses that the subject 20 may have a potential cardiac output problem 575. If not, the processor assesses if the subject 20 is being sedated in a decision step 580. If the subject is not being sedated, the processor outputs an alert that the subject 20 has potential brain damage 595. If the subject is being sedated in the decision step 580, the processor then assesses if the brain activity indicates over-sedation in a decision step 585. If so, the processor outputs an alert that the subject 20 is over sedated 590.

In some embodiments, the detected signal processing algorithms 120 may be implemented as a machine learning model.

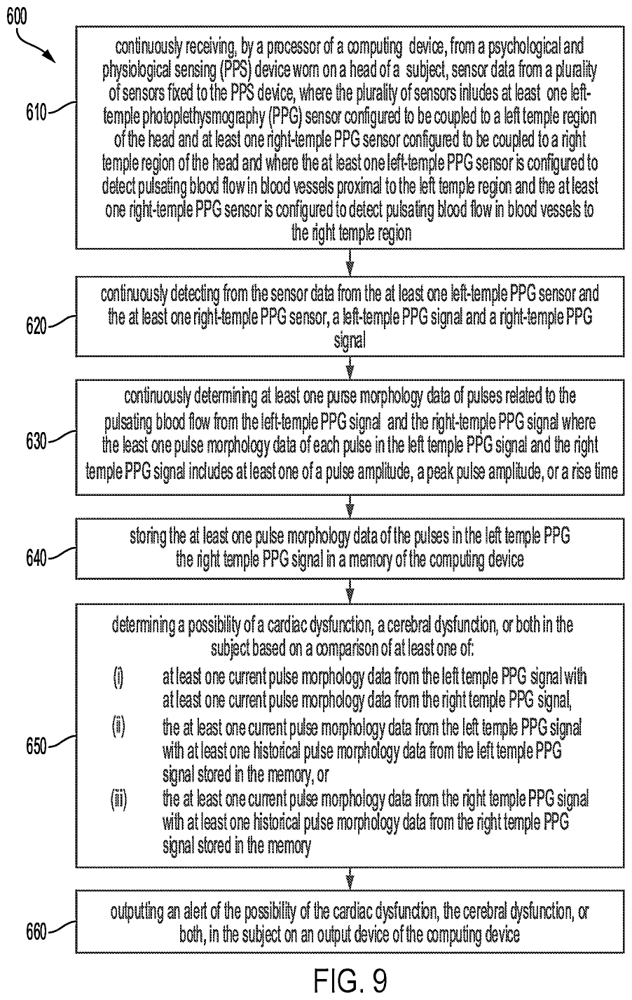

FIG. 9 is a flowchart of an exemplary method 600 for physiological and psychological parameter monitoring from a subject's head, in accordance with one or more embodiments of the present disclosure. Method 600 may be performed by any processor of the following computing devices: the processor 105 incorporated into the PPS device 10, the processor 191 from the mobile computing device 190, and/or the processor associated with the server 205 and/or the backend server 220.

Method 600 may include continuously receiving 610, by a processor of a computing device, from a psychological and physiological sensing (PPS) device worn on a head of a subject, sensor data from a plurality of sensors fixed to the PPS device, where the plurality of sensors includes at least one left-temple photoplethysmography (PPG) sensor configured to be coupled to a left temple region of the head and at least one right-temple PPG sensor configured to be coupled to a right temple region of the head and where the at least one left-temple PPG sensor is configured to detect pulsating blood flow in blood vessels proximal to the left temple region and the at least one right-temple PPG sensor is configured to detect pulsating blood flow in blood vessels to the right temple region.

Method 600 may include continuously detecting 620 from the sensor data from the at least one left-temple PPG sensor and the at least one right-temple PPG sensor, a left-temple PPG signal and a right-temple PPG signal.

Method 600 may include continuously determining 630 at least one pulse morphology data of pulses related to the pulsating blood flow from the left-temple PPG signal and the right-temple PPG signal where the least one pulse morphology data of each pulse in the left temple PPG signal and the right temple PPG signal includes at least one of a pulse amplitude of each pulse, a peak pulse amplitude of each pulse, or a rise time of each pulse.

Method 600 may include storing 640 the at least one pulse morphology data of the pulses in the left temple PPG signal and the right temple PPG signal in a memory of the computing device.

Method 600 may include determining 650 a possibility of a cardiac dysfunction, a cerebral dysfunction, or both in the subject based on a comparison of at least one of:
(i) at least one current pulse morphology data from the left temple PPG signal with at least one current pulse morphology data from the right temple PPG signal,
(ii) the at least one current pulse morphology data from the left temple PPG signal with at least one historical pulse morphology data from the left temple PPG signal stored in the memory, or
(iii) the at least one current pulse morphology data from the right temple PPG signal with at least one historical pulse morphology data from the right temple PPG signal stored in the memory.

Method 300 may include outputting 660 an alert of the possibility of the cardiac dysfunction, the cerebral dysfunction, or both, in the subject on an output device of the computing device.

In some embodiments, a method may include continuously receiving, by a processor of a computing device, from a psychological and physiological sensing (PPS) device worn on a head of a subject, sensor data from a plurality of sensors fixed to the PPS device. The computing device may communicate with the PPS device. The plurality of sensors may include at least one left-temple photoplethysmography (PPG) sensor configured to be coupled to a left temple region of the head and at least one right-temple PPG sensor configured to be coupled to a right temple region of the head. The at least one left-temple PPG sensor may be configured to detect pulsating blood flow in blood vessels proximal to the left temple region and the at least one right-temple PPG sensor may be configured to detect pulsating blood flow in blood vessels to the right temple region. A left-temple PPG signal and a right-temple PPG signal may be continuously detected by the processor from the sensor data from the at least one left-temple PPG sensor and the at least one right-temple PPG sensor. At least one pulse morphology data of pulses related to the pulsating blood flow from the left-temple PPG signal and the right-temple PPG signal may be continuously determined by the processor. The least one pulse morphology data of each pulse in the left temple PPG signal and the right temple PPG signal may include a pulse amplitude of each pulse and a timestamp of each pulse. The at least one pulse morphology data of the pulses in the left temple PPG signal and the right temple PPG signal in a memory of the computing device may be stored by the processor. A possibility of a cardiac dysfunction, a cerebral dysfunction, or both in the subject may be determined by the processor based on a comparison of at least one of:
(i) at least one current pulse morphology data from the left temple PPG signal with at least one current pulse morphology data from the right temple PPG signal,
(ii) the at least one current pulse morphology data from the left temple PPG signal with at least one historical pulse morphology data from the left temple PPG signal stored in the memory, or
(iii) the at least one current pulse morphology data from the right temple PPG signal with at least one historical pulse morphology data from the right temple PPG signal stored in the memory.
   An alert of the possibility of the cardiac dysfunction, the cerebral dysfunction, or both, in the subject may be outputted by the processor on an output device of the computing device.

In some embodiments, the output device may be a display, a speaker for generating an alarm, or both.

In some embodiments, the computing device may be selected from the group consisting of a computer, a mobile computing device, electronic processing circuitry coupled to the PPS device, and a server.

In some embodiments, the cerebral dysfunction may include a hemorrhagic stroke, or an ischemic stroke.

In some embodiments, the plurality of sensors may include at least one electroencephalogram (EEG) sensor.

In some embodiments, the method may include continuously detecting, by the processor, from the sensor data from the at least one EEG sensor, at least one EEG signal. Brain activity features from the at least one EEG signal may be continuously determined by the processor. A blood oxygen level from the sensor data from the at least one right-temple PPG sensor, at least one left-temple PPG sensor, or both may continuously determine by the processor. A possibility of brain damage, over-sedation, a cardiac output reduction, a cardiac output problem, or any combination thereof, in the subject may be determine by the processor based in part on:
(i) the comparison between the at least one current pulse morphology data from the left temple PPG signal and the at least one historical pulse morphology data from the left temple PPG signal stored in the memory,
(ii) the comparison between the at least one current pulse morphology data from the right temple PPG signal and the at least one historical pulse morphology data from the right temple PPG signal stored in the memory,
(iii) the brain activity features, and
(iv) the blood oxygen level.
   An alert of the possibility of brain damage, over-sedation, the cardiac output reduction, the cardiac output problem, or any combination thereof in the subject on the output device of the computing device may be outputted by the processor.

In some embodiments, the plurality of sensors may include at least one left-temple electrocardiogram (ECG) sensor (e.g., see the ECG sensor 170 of FIG. 2) configured to be coupled to the left temple region and at least one right-temple ECG sensor (e.g., see the ECG sensor 170 of FIG. 2) configured to be coupled to the right temple region of the head.

In some embodiments, the method may include continuously detecting, by the processor, an ECG signal based on a difference between the sensor data from the at least one left-temple ECG sensor and the at least one right-temple ECG sensor. At least one ECG morphology in the ECG signal may be continuously determined by the processor. The at least one ECG morphology data may include a timestamp of each QRS complex in the ECG signal. A velocity of the pulsating blood flow in the blood vessels proximal to the left temple region based in part on a difference between the timestamp of a current QRS in the ECG signal and the timestamp of a current pulse in the left temple PPG signal may compute by the processor. A velocity of the pulsating blood flow in the blood vessels proximal to the right temple region based in part on a difference between the timestamp of a current QRS complex in the ECG signal and the timestamp of a current pulse in the right-temple PPG signal may compute by the processor.

In some embodiments, the plurality of sensors may include an accelerometer.

In some embodiments, the method may include compensating, by the processor, for noise in the left-temple PPG signal and the right-temple PPG signal caused by movements of the subject by using the output data of the accelerometer.

In some embodiments, a system may include a computing device and a psychological and physiological sensing (PPS) device worn on a head of a subject comprising a plurality of sensors fixed to the PPS device. The plurality of sensors may include at least one left-temple photoplethysmography (PPG) sensor configured to be coupled to a left temple region of the head and at least one right-temple PPG sensor configured to be coupled to a right temple region of the head. The at least one left-temple PPG sensor may be configured to detect pulsating blood flow in blood vessels proximal to the left temple region and the at least one right-temple PPG sensor may be configured to detect pulsating blood flow in blood vessels to the right temple region. The computing device may include a memory, an output device, and a processor. The processor may be configured to execute software code stored in the memory that causes the processor to continuously receive sensor data from the plurality of sensors, where the computing device may communicate with the PPS device, continuously detect from the sensor data from the at least one left-temple PPG sensor and the at least one right-temple PPG sensor, a left-temple PPG signal and a right-temple PPG signal, continuously determine at least one pulse morphology data of pulses related to the pulsating blood flow from the left-temple PPG signal and the right-temple PPG signal, where the least one pulse morphology data of each pulse in the left temple PPG signal and the right temple PPG signal may include at least one of a pulse amplitude of each pulse, a peak pulse amplitude of each pulse, or a rise time of each pulse, store the at least one pulse morphology data of the pulses in the left temple PPG signal and the right temple PPG signal in the memory, determine a possibility of a cardiac dysfunction, a cerebral dysfunction, or both in the subject based on a comparison of at least one of:
at least one current pulse morphology data from the left temple PPG signal with at least one current pulse morphology data from the right temple PPG signal,
(ii) the at least one current pulse morphology data from the left temple PPG signal with at least one historical pulse morphology data from the left temple PPG signal stored in the memory, or
(iii) the at least one current pulse morphology data from the right temple PPG signal with at least one historical pulse morphology data from the right temple PPG signal stored in the memory, and
   output an alert of the possibility of the cardiac dysfunction, the cerebral dysfunction, or both, in the subject on the output device.

In some embodiments, the output device may be a display, a speaker for generating an alarm, or both.

In some embodiments, the computing device may be selected from the group consisting of a computer, a mobile computing device, electronic processing circuitry coupled to the PPS device, and a server.

In some embodiments, the cerebral dysfunction may include a hemorrhagic stroke, or an ischemic stroke.

In some embodiments, the plurality of sensors may include at least one electroencephalogram (EEG) sensor.

In some embodiments. the processor may be further configured to:
continuously detect from the sensor data from the at least one EEG sensor, at least one EEG signal;
continuously determine brain activity features from the at least one EEG signal;
continuously determine a blood oxygen level from the sensor data from the at least one right-temple PPG sensor, at least one left-temple PPG sensor, or both;
determine a possibility of brain damage, over-sedation, a cardiac output reduction, a cardiac output problem, or any combination thereof, in the subject based in part on:
   (i) the comparison between the at least one current pulse morphology data from the left temple PPG signal and the at least one historical pulse morphology data from the left temple PPG signal stored in the memory,
   (ii) the comparison between the at least one current pulse morphology data from the right temple PPG signal and the at least one historical pulse morphology data from the right temple PPG signal stored in the memory,
   (iii) the brain activity features, and
   (iv) the blood oxygen level; and
output an alert of the possibility of brain damage, over-sedation, the cardiac output reduction, the cardiac output problem, or any combination thereof in the subject on the output device.

In some embodiments, the plurality of sensors may include at least one left-temple electrocardiogram (ECG) sensor configured to be coupled to the left temple region and at least one right-temple ECG sensor configured to be coupled to the right temple region of the head.

In some embodiments, the processor may be further configured to:
continuously detect an ECG signal based on a difference between the sensor data from the at least one left-temple ECG sensor and the at least one right-temple ECG sensor;
continuously determine at least one ECG morphology data in the ECG signal;
   where the at least one ECG morphology data may include a timestamp of each QRS complex in the ECG signal;
compute a velocity of the pulsating blood flow in the blood vessels proximal to the left temple region based in part on a difference between the timestamp of a current QRS complex in the ECG signal and the timestamp of a current pulse in the left temple PPG signal; and
compute a velocity of the pulsating blood flow in the blood vessels proximal to the right temple region based in part on a difference between the timestamp of a current QRS complex in the ECG signal and the timestamp of a current pulse in the right-temple PPG signal.

In some embodiments, the plurality of sensors may include an accelerometer.

In some embodiments, the processor may be further configured to compensate for noise in the left-temple PPG signal and the right-temple PPG signal caused by movements of the subject by using the output data of the accelerometer.

In some embodiments, the PPS device may further include a bridge of an adjustable length with a first end fixed to the at least one left-temple sensor and a second end fixed to the at least one right-temple sensor. The adjustable length may ensure that the at least one left-temple sensor is positioned over the left temple of the subject and the at least one right-temple sensor is positioned over the right temple of the subject, respectively.

In some embodiments, the at least one left-temple sensor and the at least one right-temple sensor may each include electrodes for contacting the left temple and the right temple respectively of the subject.

In some embodiments, the PPS device may further include an electronic circuitry housing fixed to the bridge and comprising electronic circuitry.

In some embodiments, the bridge may include a lumen. The at least one left-temple sensor and the at least one right-temple sensor may be electrically coupled to the electronic circuitry by wires within the lumen.

In some embodiments, the plurality of sensors may include at least one electroencephalogram (EEG) sensor coupled to a forehead of the subject. A cable may electrically couple the at least one EEG sensor to the electronic circuitry in the electronic circuitry housing.

In some embodiments, the PPS device may further include a power unit. A cable may electrically couple the power unit to the electronic circuitry to enable the power unit to power the electronic circuitry.

In some embodiments, exemplary inventive, specially programmed computing systems/platforms with associated devices are configured to operate in the distributed network environment, communicating with one another over one or more suitable data communication networks such as communication network 210 (e.g., the Internet, satellite, etc.) and utilizing one or more suitable data communication protocols/modes such as, without limitation, IPX/SPX, X.25, AX.25, AppleTalk™, TCP/IP (e.g., HTTP), near-field wireless communication (NFC), RFID, Narrow Band Internet of Things (NBIOT), 3G, 4G, 5G, GSM, GPRS, WiFi, WiMax, CDMA, satellite, ZigBee, and other suitable communication modes. In some embodiments, the NFC can represent a short-range wireless communications technology in which NFC-enabled devices are “swiped,” “bumped,” “tap” or otherwise moved in close proximity to communicate. In some embodiments, the NFC could include a set of short-range wireless technologies, typically requiring a distance of 10 cm or less. In some embodiments, the NFC may operate at 13.56 MHz on ISO/IEC 18000-3 air interface and at rates ranging from 106 kbit/s to 424 kbit/s. In some embodiments, the NFC can involve an initiator and a target; the initiator actively generates an RF field that can power a passive target. In some embodiments, this can enable NFC targets to take very simple form factors such as tags, stickers, key fobs, or cards that do not require batteries. In some embodiments, the NFC's peer-to-peer communication can be conducted when a plurality of NFC-enable devices (e.g., smartphones) within close proximity of each other.

In some embodiments, input/output devices 185 and/or 193 may also include a number of external or internal devices such as a mouse, a CD-ROM, DVD, a physical or virtual keyboard, a display, a speaker, or other input or output devices.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

Examples of hardware elements in PPS device 10, mobile device 190, server 200 and/or backend server 220 may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, such as system 100, as used herein, include any combination of hardware and software. Examples of software may include software components, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, etc.).

In some embodiments, one or more of exemplary inventive computer-based systems/platforms, exemplary inventive computer-based devices, and/or exemplary inventive computer-based components of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

As used herein, the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

In some embodiments, as detailed herein, one or more of exemplary inventive computer-based systems/platforms, exemplary inventive computer-based devices, and/or exemplary inventive computer-based components of the present disclosure may obtain, manipulate, transfer, store, transform, generate, and/or output any digital object and/or data unit (e.g., from inside and/or outside of a particular application) that can be in any suitable form such as, without limitation, a file, a contact, a task, an email, a social media post, a map, an entire application (e.g., a calculator), etc. In some embodiments, as detailed herein, one or more of exemplary inventive computer-based systems/platforms, exemplary inventive computer-based devices, and/or exemplary inventive computer-based components of the present disclosure may be implemented across one or more of various computer platforms such as, but not limited to: (1) FreeBSD, NetBSD, OpenBSD; (2) Linux; (3) Microsoft Windows; (4) OS X (MacOS); (5) MacOS 11; (6) Solaris; (7) Android; (8) iOS; (9) Embedded Linux; (10) Tizen; (11) WebOS; (12) IBM i; (13) IBM AIX; (14) Binary Runtime Environment for Wireless (BREW); (15) Cocoa (API); (16) Cocoa Touch; (17) Java Platforms; (18) JavaFX; (19) JavaFX Mobile; (20) Microsoft DirectX; (21) .NET Framework; (22) Silverlight; (23) Open Web Platform; (24) Oracle Database; (25) Qt; (26) Eclipse Rich Client Platform; (27) SAP NetWeaver; (28) Smartface; and/or (29) Windows Runtime.

In some embodiments, exemplary inventive computer-based systems/platforms, exemplary inventive computer-based devices, and/or exemplary inventive computer-based components of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

In some embodiments, exemplary inventive computer-based systems/platforms, exemplary inventive computer-based devices, and/or exemplary inventive computer-based components of the present disclosure may be configured to handle numerous concurrent subjects or users that may be, but is not limited to, at least 100 (e.g., but not limited to, 100-999), at least 1,000 (e.g., but not limited to, 1,000-9,999), at least 10,000 (e.g., but not limited to, 10,000-99,999), at least 100,000 (e.g., but not limited to, 100,000-999,999), at least 1,000,000 (e.g., but not limited to, 1,000,000-9,999,999), at least 10,000,000 (e.g., but not limited to, 10,000,000-99,999,999), at least 100,000,000 (e.g., but not limited to, 100,000,000-999,999,999), at least 1,000,000,000 (e.g., but not limited to, 1,000,000,000-999,999,999,999), and so on.

As used herein, the term “mobile electronic device,” or “mobile computing device” or the like, may refer to any portable electronic device that may or may not be enabled with location tracking functionality (e.g., MAC address, Internet Protocol (IP) address, or the like). For example, a mobile electronic device can include, but is not limited to, a mobile phone, Personal Digital Assistant (PDA), Blackberry™, Pager, Smartphone, or any other reasonable mobile electronic device.

As used herein, the terms “proximity detection,” “locating,” “location data,” “location information,” and “location tracking” refer to any form of location tracking technology or locating method that can be used to provide a location of, for example, a particular computing device/system/platform of the present disclosure and/or any associated computing devices, based at least in part on one or more of the following techniques/devices, without limitation: accelerometer(s), gyroscope(s), Global Positioning Systems (GPS); GPS accessed using Bluetooth™; GPS accessed using any reasonable form of wireless and/or non-wireless communication; WiFi™ server location data; Bluetooth™ based location data; triangulation such as, but not limited to, network based triangulation, WiFi™ server information based triangulation, Bluetooth™ server information based triangulation; Cell Identification based triangulation, Enhanced Cell Identification based triangulation, Uplink-Time difference of arrival (U-TDOA) based triangulation, Time of arrival (TOA) based triangulation, Angle of arrival (AOA) based triangulation; techniques and systems using a geographic coordinate system such as, but not limited to, longitudinal and latitudinal based, geodesic height based, Cartesian coordinates based; Radio Frequency Identification such as, but not limited to, Long range RFID, Short range RFID; using any form of RFID tag such as, but not limited to active RFID tags, passive RFID tags, battery assisted passive RFID tags; or any other reasonable way to determine location. For ease, at times the above variations are not listed or are only partially listed; this is in no way meant to be a limitation.

As used herein, the terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

In some embodiments, the exemplary inventive computer-based systems/platforms, the exemplary inventive computer-based devices, and/or the exemplary inventive computer-based components of the present disclosure may be configured to securely store and/or transmit data by utilizing one or more of encryption techniques (e.g., private/public key pair, Triple Data Encryption Standard (3DES), block cipher algorithms (e.g., IDEA, RC2, RCS, CAST and Skipjack), cryptographic hash algorithms (e.g., MD5, RIPEMD-160, RTR0, SHA-1, SHA-2, Tiger (TTH), WHIRLPOOL, RNGs).

The aforementioned examples are, of course, illustrative and not restrictive.

In some embodiments, the exemplary inventive computer-based systems/platforms, the exemplary inventive computer-based devices, and/or the exemplary inventive computer-based components of the present disclosure (e.g., detected signal processing algorithm module 120) may be configured to utilize one or more exemplary AI/machine learning techniques chosen from, but not limited to, decision trees, boosting, support-vector machines, neural networks, nearest neighbor algorithms, Naive Bayes, bagging, random forests, and the like. In some embodiments and, optionally, in combination of any embodiment described above or below, an exemplary neutral network technique may be one of, without limitation, feedforward neural network, radial basis function network, recurrent neural network, convolutional network (e.g., U-net) or other suitable network. In some embodiments and, optionally, in combination of any embodiment described above or below, an exemplary implementation of Neural Network may be executed as follows:
i) Define Neural Network architecture/model,
ii) Transfer the input data to the exemplary neural network model,
iii) Train the exemplary model incrementally,
iv) determine the accuracy for a specific number of timesteps,
v) apply the exemplary trained model to process the newly-received input data,
vi) optionally and in parallel, continue to train the exemplary trained model with a predetermined periodicity.

In some embodiments and, optionally, in combination of any embodiment described above or below, the exemplary trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the exemplary trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the exemplary aggregation function may be a mathematical function that combines (e.g., sum, product, etc.) input signals to the node. In some embodiments and, optionally, in combination of any embodiment described above or below, an output of the exemplary aggregation function may be used as input to the exemplary activation function. In some embodiments and, optionally, in combination of any embodiment described above or below, the bias may be a constant value or function that may be used by the aggregation function and/or the activation function to make the node more or less likely to be activated.

Publications cited throughout this document are hereby incorporated by reference in their entirety. While one or more embodiments of the present disclosure have been described, it is understood that these embodiments are illustrative only, and not restrictive, and that many modifications may become apparent to those of ordinary skill in the art, including that various embodiments of the inventive methodologies, the inventive systems/platforms, and the inventive devices described herein can be utilized in any combination with each other. Further still, the various steps may be carried out in any desired order (and any desired steps may be added and/or any desired steps may be eliminated).

1. A method, comprising:
continuously receiving, by a processor of a computing device, from a psychological and physiological sensing (PPS) device worn on a head of a subject, sensor data from a plurality of sensors fixed to the PPS device;
wherein the computing device communicates with the PPS device;
wherein the plurality of sensors comprises at least one left-temple photoplethysmography (PPG) sensor configured to be coupled to a left-temple region of the head and at least one right-temple PPG sensor configured to be coupled to a right-temple region of the head;
wherein the at least one left-temple PPG sensor is configured to detect pulsating blood flow in blood vessels proximal to the left-temple region and the at least one right-temple PPG sensor is configured to detect pulsating blood flow in blood vessels to the right-temple region;
continuously detecting, by the processor, from the sensor data from the at least one left-temple PPG sensor and the at least one right-temple PPG sensor, a left-temple PPG signal and a right-temple PPG signal;
continuously determining, by the processor, at least one pulse morphology data of pulses related to the pulsating blood flow from the left-temple PPG signal and the right-temple PPG signal;
wherein the least one pulse morphology data of each pulse in the left-temple PPG signal and the right-temple PPG signal comprises at least one of:
(i) a pulse amplitude of each pulse,
(ii) a peak pulse amplitude of each pulse, or
(iii) a rise time of each pulse;
storing, by the processor, the at least one pulse morphology data of the pulses in the left-temple PPG signal and the right-temple PPG signal in a memory of the computing device;
determining, by the processor, a possibility of a cardiac dysfunction, a cerebral dysfunction, or both in the subject based on a comparison of at least one of:
(i) at least one current pulse morphology data from the left-temple PPG signal with at least one current pulse morphology data from the right-temple PPG signal,
(ii) the at least one current pulse morphology data from the left-temple PPG signal with at least one historical pulse morphology data from the left-temple PPG signal stored in the memory, or
(iii) the at least one current pulse morphology data from the right-temple PPG signal with at least one historical pulse morphology data from the right-temple PPG signal stored in the memory; and
outputting, by the processor, an alert of the possibility of the cardiac dysfunction, the cerebral dysfunction, or both, in the subject on an output device of the computing device.

2. The method according to claim 1, wherein the output device is a display, a speaker for generating an alarm, or both.

3. The method according to claim 1, wherein the computing device is selected from the group consisting of a computer, a mobile computing device, electronic processing circuitry coupled to the PPS device, and a server.

4. The method according to claim 1, wherein the cerebral dysfunction comprises a hemorrhagic stroke, or an ischemic stroke.

5. The method according to claim 1, wherein the plurality of sensors comprises at least one electroencephalogram (EEG) sensor.

6. The method according to claim 5, further comprising:
continuously detecting, by the processor, from the sensor data from the at least one EEG sensor, at least one EEG signal;
continuously determining, by the processor, brain activity features from the at least one EEG signal;
continuously determining, by the processor, a blood oxygen level from the sensor data from the at least one right-temple PPG sensor, at least one left-temple PPG sensor, or both;
determining, by the processor, a possibility of brain damage, over-sedation, a cardiac output reduction, a cardiac output problem, or any combination thereof, in the subject based in part on:
(i) the comparison between the at least one current pulse morphology data from the left-temple PPG signal and the at least one historical pulse morphology data from the left-temple PPG signal stored in the memory,
(ii) the comparison between the at least one current pulse morphology data from the right-temple PPG signal and the at least one historical pulse morphology data from the right-temple PPG signal stored in the memory,
(iii) the brain activity features, and
(iv) the blood oxygen level; and
outputting, by the processor, an alert of the possibility of brain damage, over-sedation, the cardiac output reduction, the cardiac output problem, or any combination thereof in the subject on the output device of the computing device.

7. The method according to claim 1, wherein the plurality of sensors comprises at least one left-temple electrocardiogram (ECG) sensor configured to be coupled to the left-temple region and at least one right-temple ECG sensor configured to be coupled to the right-temple region of the head.

8. The method according to claim 7, further comprising:
continuously detecting, by the processor, an ECG signal from a difference between the sensor data from the at least one left-temple ECG sensor and the at least one right-temple ECG sensor;
continuously determining, by the processor, at least one ECG morphology data in the ECG data;
wherein the at least one ECG morphology data comprises a timestamp of each QRS complex;
computing, by the processor, a velocity of the pulsating blood flow in the blood vessels proximal to the left-temple region based in part on a difference between the timestamp of a current QRS complex in the ECG signal and the timestamp of a current pulse in the left-temple PPG signal; and
computing, by the processor, a velocity of the pulsating blood flow in the blood vessels proximal to the right-temple region based in part on a difference between the timestamp of a current QRS in the ECG signal and the timestamp of a current pulse in the right-temple PPG signal.

9. The method according to claim 1, wherein the plurality of sensors comprises an accelerometer.

10. The method according to claim 9, further comprising compensating, by the processor, for noise in the left-temple PPG signal and the right-temple PPG signal caused by movements of the subject by using the output data of the accelerometer.

11. A system, comprising:
a psychological and physiological sensing (PPS) device worn on a head of a subject comprising a plurality of sensors fixed to the PPS device;
wherein the plurality of sensors comprises at least one left-temple photoplethysmography (PPG) sensor configured to be coupled to a left-temple region of the head and at least one right-temple PPG sensor configured to be coupled to a right-temple region of the head;
wherein the at least one left-temple PPG sensor is configured to detect pulsating blood flow in blood vessels proximal to the left-temple region and the at least one right-temple PPG sensor is configured to detect pulsating blood flow in blood vessels to the right-temple region;
a computing device comprising a memory, an output device, and a processor, wherein the processor is configured to execute software code stored in the memory that causes the processor to:
continuously receive sensor data from the plurality of sensors;
wherein the computing device communicates with the PPS device;
continuously detect from the sensor data from the at least one left-temple PPG sensor and the at least one right-temple PPG sensor, a left-temple PPG signal and a right-temple PPG signal;
continuously determine at least one pulse morphology data of pulses related to the pulsating blood flow from the left-temple PPG signal and the right-temple PPG signal;
wherein the least one pulse morphology data of each pulse in the left-temple PPG signal and the right-temple PPG signal comprises at least one of:
(i) a pulse amplitude of each pulse,
(ii) a peak pulse amplitude of each pulse, or
(iii) a rise time of each pulse;
store the at least one pulse morphology data of the pulses in the left-temple PPG signal and the right-temple PPG signal in the memory;
determine a possibility of a cardiac dysfunction, a cerebral dysfunction, or both in the subject based on a comparison of at least one of:
(i) at least one current pulse morphology data from the left-temple PPG signal with at least one current pulse morphology data from the right-temple PPG signal,
(ii) the at least one current pulse morphology data from the left-temple PPG signal with at least one historical pulse morphology data from the left-temple PPG signal stored in the memory, or
(iii) the at least one current pulse morphology data from the right-temple PPG signal with at least one historical pulse morphology data from the right-temple PPG signal stored in the memory; and
output an alert of the possibility of the cardiac dysfunction, the cerebral dysfunction, or both, in the subject on the output device.

12. The system according to claim 11, wherein the output device is a display, a speaker for generating an alarm, or both.

13. The system according to claim 11, wherein the computing device is selected from the group consisting of a computer, a mobile computing device, electronic processing circuitry coupled to the PPS device, and a server.

14. The system according to claim 11, wherein the cerebral dysfunction comprises a hemorrhagic stroke, or an ischemic stroke.

15. The system according to claim 11, wherein the plurality of sensors comprises at least one electroencephalogram (EEG) sensor.

16. The system according to claim 15, wherein the processor is further configured to:
continuously detect from the sensor data from the at least one EEG sensor, at least one EEG signal;
continuously determine brain activity features from the at least one EEG signal;
continuously determine a blood oxygen level from the sensor data from the at least one right-temple PPG sensor, at least one left-temple PPG sensor, or both;
determine a possibility of brain damage, over-sedation, a cardiac output reduction, a cardiac output problem, or any combination thereof, in the subject based in part on:
(i) the comparison between the at least one current pulse morphology data from the left-temple PPG signal and the at least one historical pulse morphology data from the left-temple PPG signal stored in the memory,
(ii) the comparison between the at least one current pulse morphology data from the right-temple PPG signal and the at least one historical pulse morphology data from the right-temple PPG signal stored in the memory,
(iii) the brain activity features, and
(iv) the blood oxygen level; and
output an alert of the possibility of brain damage, over-sedation, the cardiac output reduction, the cardiac output problem, or any combination thereof in the subject on the output device.

17. The system according to claim 11, wherein the plurality of sensors comprises at least one left-temple electrocardiogram (ECG) sensor configured to be coupled to the left-temple region and at least one right-temple ECG sensor configured to be coupled to the right-temple region of the head.

18. The system according to claim 17, wherein the processor is further configured to:
continuously detect an ECG signal based on a difference between the sensor data from the at least one left-temple ECG sensor and the at least one right-temple ECG sensor;
continuously determine at least one ECG morphology data in the ECG signal;
wherein the at least one ECG morphology data comprises a timestamp of each QRS complex in the ECG signal;
compute a velocity of the pulsating blood flow in the blood vessels proximal to the left-temple region based in part on a difference between the timestamp of a current QRS complex in the ECG signal and the timestamp of a current pulse in the left-temple PPG signal; and
compute a velocity of the pulsating blood flow in the blood vessels proximal to the right-temple region based in part on a difference between the timestamp of a current QRS complex in the ECG signal and the timestamp of a current pulse in the right-temple PPG signal.

19. The system according to claim 11, wherein the plurality of sensors comprises an accelerometer.

20. The system according to claim 19, wherein the processor is further configured to compensate for noise in the left-temple PPG signal and the right-temple PPG signal caused by movements of the subject by using the output data of the accelerometer.

21. The system according to claim 11, wherein the PPS device further comprises a bridge of an adjustable length with a first end fixed to the at least one left-temple sensor and a second end fixed to the at least one right-temple sensor; and
wherein the adjustable length ensures that the at least one left-temple sensor is positioned over the left temple of the subject and the at least one right-temple sensor is positioned over the right temple of the subject, respectively.

22. The system according to claim 21, wherein the at least one left-temple sensor and the at least one right-temple sensor each comprise electrodes for contacting the left temple and the right temple respectively of the subject.

23. The system according to claim 21, wherein the PPS device further comprises an electronic circuitry housing fixed to the bridge and comprising electronic circuitry.

24. The system according to claim 23, wherein the bridge comprises a lumen; and
wherein the at least one left-temple sensor and the at least one right-temple sensor are electrically coupled to the electronic circuitry by wires within the lumen.

25. The system according to claim 23, wherein the plurality of sensors comprises at least one electroencephalogram (EEG) sensor coupled to a forehead of the subject; and
wherein a cable electrically couples the at least one EEG sensor to the electronic circuitry in the electronic circuitry housing.

26. The system according to claim 23, wherein the PPS device further comprises a power unit; and wherein a cable electrically couples the power unit to the electronic circuitry to enable the power unit to power the electronic circuitry.
